(19) United States
(12) Patent Application Publication
(10) Pub. No.: US 2004/0208915 A1
(43) Pub. Date: Oct. 21, 2004

(54) Transdermal administration device for animals

(76) Inventors: Nick Abbott, Sheffield (GB); Jonathan Bowtell, Sheffield (GB)

Correspondence Address: ROSENTHAL & OSHA L.L.P., Suite 2800, 1221 McKinney Street, Houston, TX, 77010, US

(21) Appl. No.: 10/414,251

(22) Filed: Apr. 15, 2003

(51) Int. Cl. A61K 35/20; A61K 9/70

(52) U.S. Cl. 424/449; 424/535

(57) ABSTRACT

A device for the transdermal administration of a substance to an animal comprising: positioning means and delivery means. The positioning means comprising an inner and an outer surface and being configured to position and hold in place said delivery means. The delivery means comprising an inner and an outer surface and being configured to facilitate the transdermal administration of substances. The outer surface of said delivery means and said inner surface of said positioning means are attachable to each other.

### FIELD OF THE INVENTION

The present invention relates to the transdermal administration of substances to animals, wherein a delivery means impregnated with a substance is brought into proximity with the skin of an animal and held in this position by a positioning means.

### BACKGROUND TO THE INVENTION

Known means of administering substances to animals generally consist of either administering such substances by direct oral submission (or mixing the substance with a food stuff) or alternatively by injection into the animal. The administration of such substances to animals may have many beneficial effects and cause an increase in the welfare of the animal and an increase in the quality of interaction with its owner.

A third method of substance administration used in addition to those previously described for humans is the transdermal administration of substances for instance in the form of a patch. Such patches are used to administer a wide range of medicinal and other types of substances transdermally, transdermal administration has the benefit of not requiring a substance to be swallowed orally which may prove difficult if the substance is distasteful or the amount of substance to be administered is large and is also less stressful and without pain in comparison to the administration of substances by intra-dermal or intra-muscular injection.

The incorporation of medicinal or other substances into a collar or other form of restraining device have previously been described for instance U.S. Pat. No. 5,146,875, such prior art apparatus however, are only intended to apply such substances externally for example the administration of anti-parasitic or insecticides to control fleas, ticks or other parasites. Such prior art collars are impregnated with chemicals designed to persist for an extremely long time, to give long term protection against fleas, ticks or other parasites and are therefore not designed to facilitate the easy changing of the impregnated sections of the device if in fact these are separate from the restraining device itself, in many cases they are integral. As is well known from human patches given the volatile nature of the components found within transdermal patches, they must be changed on a regular basis and most normally every one to two days. Such prior art restraining devices therefore would need to be changed every one to two days making them prohibitively expensive for their intended customers or very inconvenient to use.

As a consequence of the forgoing situation, there has existed a need among a significant number of pet owners for a new type of device that will allow the effective transdermal administration of substances to their animals and the provision of such a device is the stated objective of the present invention.

### SUMMARY OF THE INVENTION

According to a first aspect there is provided a device for the transdermal administration of substances to animals comprising:

positioning means and delivery means:

said positioning means comprising an inner and an outer surface and being configured to position and hold in place said delivery means:

said delivery means comprising an inner and an outer surface and being configured to facilitate the transdermal administration of said substances;

said outer surface of said delivery means being configured to attach to said inner surface of said positioning means.

Preferably said positioning means comprises a strap.

Preferably said positioning means comprises a strap and said delivery means comprises a replaceable impregnated cassette.

Preferably said positioning means comprises a strap and said delivery means comprises a replaceable cassette impregnated with colostrum.

Preferably said positioning means comprises a strap and said delivery means comprises a replaceable cassette impregnated with a colostrum derivative.

Preferably said positioning means comprises a strap and said delivery means comprises a replaceable cassette impregnated with a multi-vitamin and mineral solution.

Alternatively said positioning means comprises a strap and said delivery means is configured to form a reservoir when held in place by said positioning means;

said reservoir being configured to accept an impregnated cassette.

Preferably said positioning means comprises a strap and wherein said delivery means is configured to form a reservoir when held in place by said positioning means;

said reservoir being configured to accept an impregnated cassette;

wherein said cassette is impregnated with colostrum.

Preferably said positioning means comprises a strap and wherein said delivery means is configured to form a reservoir when held in place by said positioning means;

said reservoir being configured to accept an impregnated cassette;

wherein said cassette is impregnated with a colostrum derivative.

Preferably said positioning means comprises a strap and wherein said delivery means is configured to form a reservoir when held in place by said positioning means;

said reservoir being configured to accept an impregnated cassette;

wherein said cassette is impregnated with a multi-vitamin and mineral solution

Alternatively said positioning means comprises a strap and said delivery means comprise an impermeable substrate and an adhesive matrix comprising said substances;

wherein said adhesive matrix is configured to adhere to the lowest surface of said impermeable substrate and the outer surface of said impermeable substrate is configured to adhere to the inner surface of said positioning means.

Preferably said positioning means comprises a strap and said delivery means comprise an impermeable substrate and an adhesive matrix comprising said substances;

wherein said adhesive matrix is configured to adhere to the lowest surface of said impermeable substrate and the outer surface of said impermeable substrate is configured to adhere to the inner surface of said positioning means;

wherein said adhesive matrix is impregnated with colostrum.

Preferably said positioning means comprises a strap and said delivery means comprise an impermeable substrate and an adhesive matrix comprising said substances;

wherein said adhesive matrix is configured to adhere to the lowest surface of said impermeable substrate and the outer surface of said impermeable substrate is configured to adhere to the inner surface of said positioning means;

wherein said adhesive matrix is impregnated with a colostrum derivative.

Preferably said positioning means comprises a strap and said delivery means comprise an impermeable substrate and an adhesive matrix comprising said substances;

wherein said adhesive matrix is configured to adhere to the lowest surface of said impermeable substrate and the outer surface of said impermeable substrate is configured to adhere to the inner surface of said positioning means;

wherein said adhesive matrix is impregnated with multi-vitamin and mineral solution.

Alternatively said positioning means comprises a collar.

Preferably positioning means comprises a collar and said delivery means comprises a replaceable impregnated cassette.

Preferably said positioning means comprises a collar and said delivery means comprises a replaceable cassette impregnated with colostrum.

Preferably said positioning means comprises a collar and said delivery means comprises a replaceable cassette impregnated with a colostrum derivative.

Preferably said positioning means comprises a collar and said delivery means comprises a replaceable cassette impregnated with a multi-vitamin and mineral solution.

Alternatively said positioning means comprises a collar and wherein said delivery means is configured to form a reservoir when held in place by said positioning means;

said reservoir being configured to accept an impregnated cassette.

Preferably said positioning means comprises a collar and wherein said delivery means is configured to form a reservoir when held in place by said positioning means;

said reservoir being configured to accept an impregnated cassette;

wherein said cassette is impregnated with colostrum.

Preferably said positioning means comprises a collar and wherein said delivery means is configured to form a reservoir when held in place by said positioning means;

said reservoir being configured to accept an impregnated cassette;

wherein said cassette is impregnated with a colostrum derivative.

Preferably said positioning means comprises a collar and wherein said delivery means is configured to form a reservoir when held in place by said positioning means;

said reservoir being configured to accept an impregnated cassette;

wherein said cassette is impregnated with a multi-vitamin and mineral solution.

Preferably said positioning means comprises a collar and said delivery means comprise an impermeable substrate and an adhesive matrix comprising said substances;

wherein said adhesive matrix is configured to adhere to the lowest surface of said impermeable substrate and the outer surface of said impermeable substrate is configured to adhere to the inner surface of said positioning means.

Preferably said positioning means comprises a collar and said delivery means comprise an impermeable substrate and an adhesive matrix comprising said substances;

wherein said adhesive matrix is configured to adhere to the lowest surface of said impermeable substrate and the outer surface of said impermeable substrate is configured to adhere to the inner surface of said positioning means:

wherein said adhesive matrix is impregnated with colostrum.

Preferably said positioning means comprises a collar and said delivery means comprise an impermeable substrate and an adhesive matrix comprising said substances;

wherein said adhesive matrix is configured to adhere to the lowest surface of said impermeable substrate and the outer surface of said impermeable substrate is configured to adhere to the inner surface of said positioning means;

wherein said adhesive matrix is impregnated with a colostrum derivative.

Preferably said positioning means comprises a collar and said delivery means comprise an impermeable substrate and an adhesive matrix comprising said substances;

Alternatively said positioning means comprises a harness.

Preferably said positioning means comprises a harness and said delivery means comprises a replaceable impregnated cassette.

Preferably said positioning means comprises a harness and said delivery means comprises a replaceable cassette impregnated with colostrum.

Preferably said positioning means comprises a harness and said delivery means comprises a replaceable cassette impregnated with a colostrum derivative.

Preferably said positioning means comprises a harness and said delivery means comprises a replaceable cassette impregnated with a multi-vitamin and mineral solution.

Alternatively said positioning means comprises a harness and wherein said delivery means is configured to form a reservoir when held in place by said positioning means;

said reservoir being configured to accept an impregnated cassette.

Preferably said positioning means comprises a harness and wherein said delivery means is configured to form a reservoir when held in place by said positioning means;

said reservoir being configured to accept an impregnated cassette;

wherein said cassette is impregnated with colostrum.

Preferably said positioning means comprises a harness and wherein said delivery means is configured to form a reservoir when held in place by said positioning means;

said reservoir being configured to accept an impregnated cassette;

wherein said cassette is impregnated with a colostrum derivative.

Alternatively said positioning means comprises a harness and wherein said delivery means is configured to form a reservoir when held in place by said positioning means;

said reservoir being configured to accept an impregnated cassette;

wherein said cassette is impregnated with a multi-vitamin and mineral solution.

Preferably said positioning means comprises a harness and said delivery means comprise an impermeable substrate and an adhesive matrix comprising said substances;

wherein said adhesive matrix is configured to adhere to the lowest surface of said impermeable substrate and the outer surface of said impermeable substrate is configured to adhere to the inner surface of said positioning means.

Preferably said positioning means comprises a harness and said delivery means comprise an impermeable substrate and an adhesive matrix comprising said substances;

wherein said adhesive matrix is configured to adhere to the lowest surface of said impermeable substrate and the outer surface of said impermeable substrate is configured to adhere to the inner surface of said positioning means;

wherein said adhesive matrix is impregnated with colostrum.

Preferably said positioning means comprises a harness and said delivery means comprise an impermeable substrate and an adhesive matrix comprising said substances:

wherein said adhesive matrix is configured to adhere to the lowest surface of said impermeable substrate and the outer surface of said impermeable substrate is configured to adhere to the inner surface of said positioning means;

wherein said adhesive matrix is impregnated with a colostrum derivative.

Preferably said positioning means comprises a harness and said delivery means comprise an impermeable substrate and an adhesive matrix comprising said substances;

According to a second aspect of the present invention there is provided a pet restraint device comprising means to transdermally administer compounds;

said means comprising a releasably mounted plate.

Preferably said pet restraint device is a collar.

Alternatively said pet restraint device is a harness.

Preferably said releasably mounted plate comprises colostrum.

Alternatively said releasably mounted plate comprises a colostrum derivative.

Alternatively said releasably mounted plate comprises a multi-vitamin and mineral solution.

Alternatively said releasably mounted plate is configured to form a compartment when mounted upon said pet restraint device;

said compartment being configured to hold a permeable cassette comprising said non-medicinal compounds.

### BRIEF DESCRIPTION OF THE DRAWINGS

For a better understanding of the invention and to show how the same may be carried into effect, there will now be described by way of example only, specific embodiments, methods and processes according to the present invention with reference to the accompanying drawings in which:

FIG. 1 shows a schematic representation of a device for the transdermal administration of substances to animals according to a first specific embodiment of the present invention in which the positioning means comprises a strap and the delivery means is a replaceable impregnated cassette.

FIG. 2 shows a schematic representation of a device for the transdermal administration of substances to animals according to a second specific embodiment of the present invention in which the positioning means comprises a strap and the delivery means is configured to form a reservoir when held in place by the positioning means the reservoir being configured to accept an impregnated cassette.

FIG. 3 shows a top down representation of the second specific embodiment shown in FIG. 2.

FIG. 4 shows a schematic representation of a device for the transdermal administration of substances to animals according to a third specific embodiment of the present invention in which the positioning means comprises a strap and the delivery means comprises an impermeable substrate and adhesive matrix comprising the substances to be transdermally administered.

FIG. 5 shows a top down representation of the third specific embodiment shown in FIG. 4.

FIG. 6 shows a schematic representation of a device for the transdermal administration of substances to animals according to a fourth specific embodiment of the present invention in which the positioning means comprises a collar and the delivery means comprises a replaceable impregnated cassette.

FIG. 7 shows a schematic representation of a device for the transdermal administration of substances to animals according to a fifth specific embodiment of the present invention in which the positioning means comprises a collar and the delivery means is configured to form a reservoir when held in place by the positioning means the reservoir being configured to accept an impregnated cassette.

FIG. 8 shows a top down representation of the fifth specific embodiment shown in FIG. 7.

FIG. 9 shows a schematic representation of a device for the transdermal administration of substances to animals according to a sixth specific embodiment of the present invention in which the positioning means comprises a collar and the delivery means comprises an impermeable substrate and adhesive matrix comprising the substances to be transdermally administered.

FIG. 10 shows a top down representation of the sixth specific embodiment shown in FIG. 9.

### DETAILED DESCRIPTION OF A SPECIFIC MODE FOR CARRYING OUT THE INVENTION

There will now be described by way of example a specific mode contemplated by the inventors. In the following description numerous specific details are set forth in order to provide a thorough understanding. It will be apparent however, to one skilled in the art, that the present invention may be practiced without limitation to these specific details. In other instances, well known methods and structures have not been described in detail so as not to unnecessarily obscure the description.

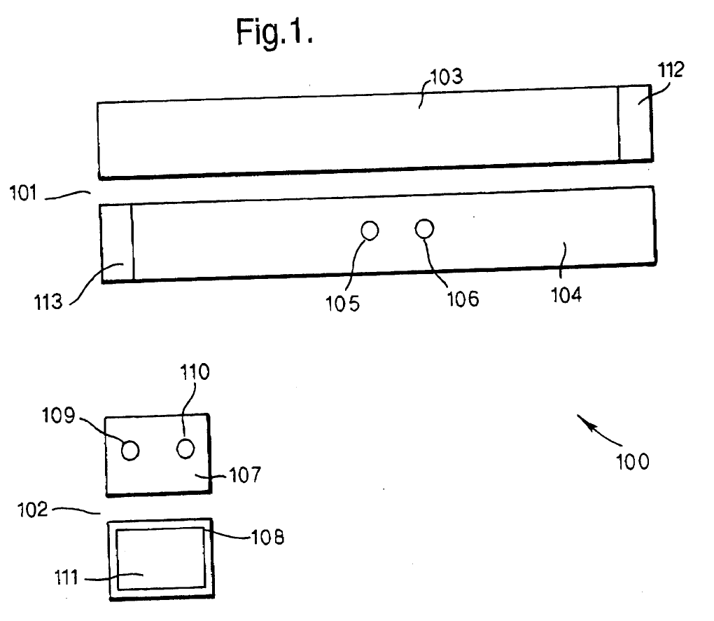

In a first specific embodiment of the invention and referring to FIG. 1 herein, a transdermal delivery device 100 comprises a positioning means 101 and a delivery means 102. The delivery means 101 is in the form of a strap and this strap further comprises an outer surface 103 and an inner surface 104. The inner surface 104 further comprises two female studs 105 and 106 these are spaced approximately 4 centimeters apart along the length of the strap 101. The delivery means 102 comprises a replaceable impregnated cassette. This impregnated replaceable cassette 102 comprises an outer surface 107 and an inner 108 surface. Upon the outer surface 107 of this impregnated replaceable cassette 102 there are two male studs 109 and 110 spaced approximately 4 centimeters apart and configured to be detachably mounted upon the inner surface 104 of said strap 101 at female studs 105 and 106. The inner surface 108 of the replaceable impregnated cassette 102 is configured so as to allow passage of substances impregnated into the replaceable cassette 102 to any animal about which the transdermal administration device has been fitted. To facilitate this administration the inner surface 108 comprises a permeable window 111 through which substances incorporated into the cassette may pass freely.

In this preferred embodiment the strap 101 is manufactured from a strong synthetic fiber such as nylon although any suitable manmade or natural material may be used. To secure the strap about the animal a first patch of Velcro® 112 is incorporated into the outer surface 103 of the strap 101 at a first end and a second patch of Velcro® 113 is incorporated into the lower surface 104 of the strap 101 at a second end. As will be obvious to those skilled in the art these two pieces of Velcro® comprising a fastening means for the strap may easily be replaced by a set of studs, buckle or any other means of fastening without affecting the character or functioning of the invention.

In operation therefore this first specific embodiment of the invention may be assembled and used as follows: assuming the strap 101 is placed upon a work surface with the outer face 103 in contact with such a surface, the user takes a replaceable impregnated cassette which has not been used before and fits this to the strap 101 by mounting the replaceable impregnated cassette 102 upon the strap 101 via the male and female studs 105, 106/109, 110 respectively. The user then takes the complete device and secures it about the animal by looping the strap 101 about the neck, torso, limb or other suitable body part, the strap is fastened in place and secured in this position when the user places the two pieces of Velcro® 112 and 113 in contact.

According to a further aspect of this first specific embodiment of the invention, the replaceable cassette 102 referred to above is impregnated with colostrum. Colostrum is the first mammary secretion that a mammal provides for its newborn in the first twenty-four to forty-eight hours following birth. It contains numerous immune system and growth factors as well as several essential nutrients and other components to give the newborn infant a physiological and immungenic boost following the trauma of birth. It is estimated that colostrum triggers at least fifty processes in the newborn infant. Several studies have shown that bovine colostrum is biologically transferable to all mammals and laboratory analysis of the immune and growth factors from bovine colostrum have been shown it to be similar to those found in a wide range of other mammals including humans. Historically, bovine colostrum has been used therapeutically in India for thousands of years and in the 20^{th} century its use was encouraged for antibiotic purposes prior to the widespread introduction of sulpha drugs and penicillin. In this first specific embodiment of the present invention the substance colostrum is impregnated into a cotton gauze material, located within the impregnated cassette 102 and is exposed to the environment via the permeable window 111 of the cassette 102. According to other specific embodiments of the present invention the substance colostrum may instead be impregnated into a water containing gel matrix or any other suitable medium for the storage and delivery of colostrum. Such a gel matrix may be formulated by the mixture of a solution or a powdered form of colostrum with a thickening ingredient comprising a natural or synthetic gel forming polymer selected from the group comprising:

gum karaya, gum acacia, locust bean gum, guar gum, modified guar gum, maltodextrin, carboxymethyl cellulose, carboxypropyl cellulose, poly-acrylamide, poly acrylic acid, a natural or synthetic rubber, a petroleum derivative, and a resin. Such a gel may be incorporated into the impregnated cassette 102 exposed to the environment via the permeable window 111 and administer substances transdermally as previously discussed.

According to a further specific aspect of this first embodiment of the present invention the replaceable impregnated cassette 102 may be impregnated with one or a combination of the known derivatives of colostrum. Colostrum as previously discussed is the first food an infant mammal receives from its mother following birth and as such is extremely important to the infant, colostrum contains many important components, these can be broken down into two major categories immune system factors and growth/nutritional factors. Colostrum is known to comprise the following:

immunoglobulins, lactoferin, transferin, lysozyme, peroxidase, praline rich 30 polypeptides, cytokines, lymphokines, oligo saccharides and glycoconjugates, thymosin, transfer factors, nucleotides and nucleosides, xanthine oxidase, growth hormone, insulin-like growth factors, epithelial growth factors, transforming growth factors A and B, fibroblast growth factor, platelet derived growth factors, trypsin inhibitors, protease inhibitors, leptin, insulin, vitamin—binding proteins, mineral binding proteins, cyclic acleosine monophosphate, enzyme inhibitors.

According to a further specific aspect of this first specific embodiment of the present invention the replaceable impregnated cassette 102 may be impregnated with a multi vitamin and mineral solution. The benefits of maintaining the recommended daily intake of the major vitamins and minerals are well known and the use of vitamin and mineral supplements is also known and an accepted way of maintaining the levels of these important substances.

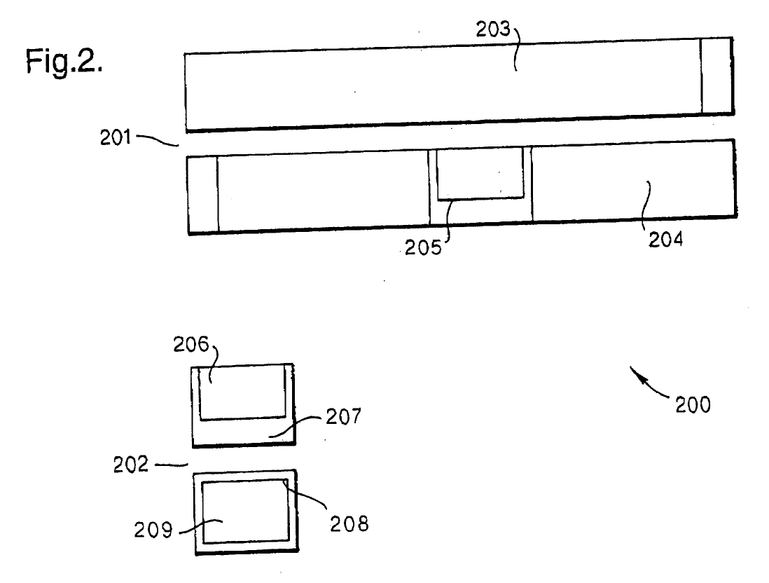

In a second specific embodiment of the present invention and referring to FIG. 2 herein, a transdermal delivery device 200 comprises a positioning means 201 and a delivery means 202. The positioning means 201 is in the form of a strap, and this strap further comprises an outer surface 203 and an inner surface 204. The inner surface 204 of the strap 201 further comprises a region of Velcro® 205, this region of Velcro® 205 is located in approximately in the middle of the length of the strap 201 and is in the shape of a U. The delivery means 202 comprises an outer surface 206 and an inner surface 208. The outer surface 206 further comprises a piece of Velcro® 207 configured to substantially adhere said delivery means 202 to said strap 201 and therefore is of similar dimensions and shape to Velcro® 205. The delivery means. 202 is configured to form a reservoir when held in place against the strap 201 by Velcro® 205 and 207. The strap 201 and delivery means are constructed of materials much as described for the first specific embodiment of the present invention.

In this second preferred embodiment of the present invention the transdermal delivery device 200 may be assembled and used in the following way:

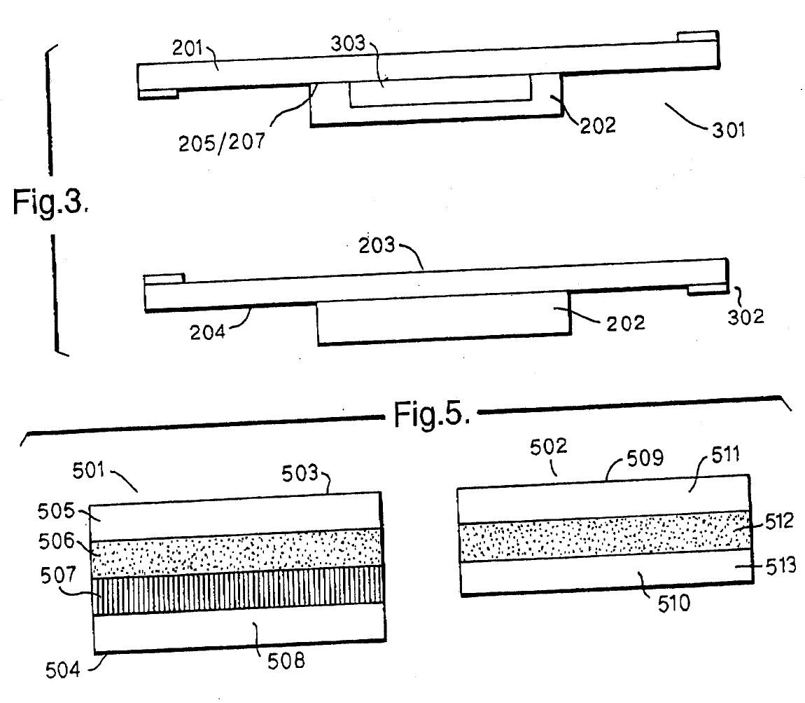

assuming the strap 201 is placed upon a flat work surface with the outer surface 203 of the strap 201 in contact with this work surface the delivery means 202 may be placed such that Velcro® 207 on the outer surface 206 of the delivery means 202 aligns with Velcro® 205 on the inner surface 204 of the strap 201. Referring to FIG. 3 which shows a top and bottom view of this second specific embodiment of this present invention, 301 shows a top down view of the present invention and 302 shows a bottom view of the second specific embodiment of the present invention. 301 shows the strap 201 attached via the Velcro® 205/207 to the delivery means 202 the configuration of the strap 201 and delivery means 202 causes a reservoir 303 to be formed by the joining of these two component parts. The delivery means 202 is configured such that an aperture is created when the strap 201 and delivery means 202 come together in this fashion, this allows an impregnated cassette to be inserted into the reservoir 303. The delivery means are further configured such that on the bottom of the transdermal delivery means 302 no aperture exists such that the impregnated cassette is held in reservoir 303 and does not fall out during use. The lower surface 208 of the delivery means 202 further comprises a permeable window 209 through which substances contained within the impregnated cassette may pass from the cassette to the recipient animal.

The transdermal delivery means may then be positioned about the animal much as previously described for the first specific embodiment of the present invention. Once the impregnated cassette has fulfilled its service life, as previously discussed this would probably be one to two days the owner of this animal may simply remove the impregnated cassette from reservoir 303 and replace with a fresh impregnated cassette to continue the administration of substances to the animal.

According to a further specific aspect of this second embodiment of the present invention the impregnated cassette may comprise colostrum, a derivative of colostrum or a multi-vitamin and mineral solution. Details of these substances are much as previously described for the first specific embodiment.

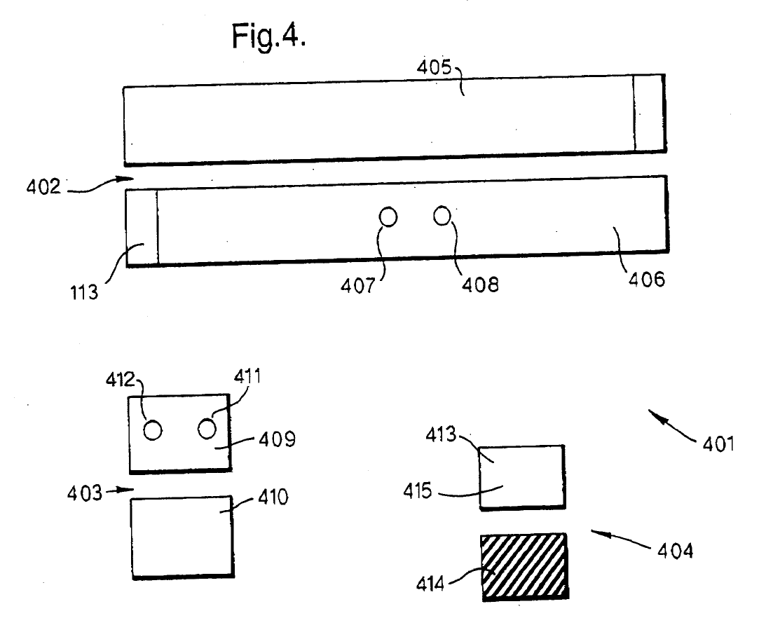

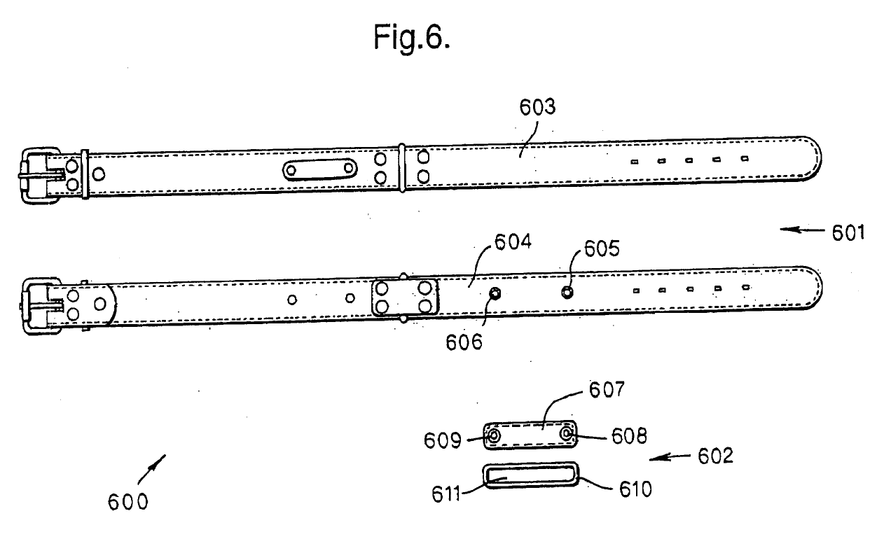

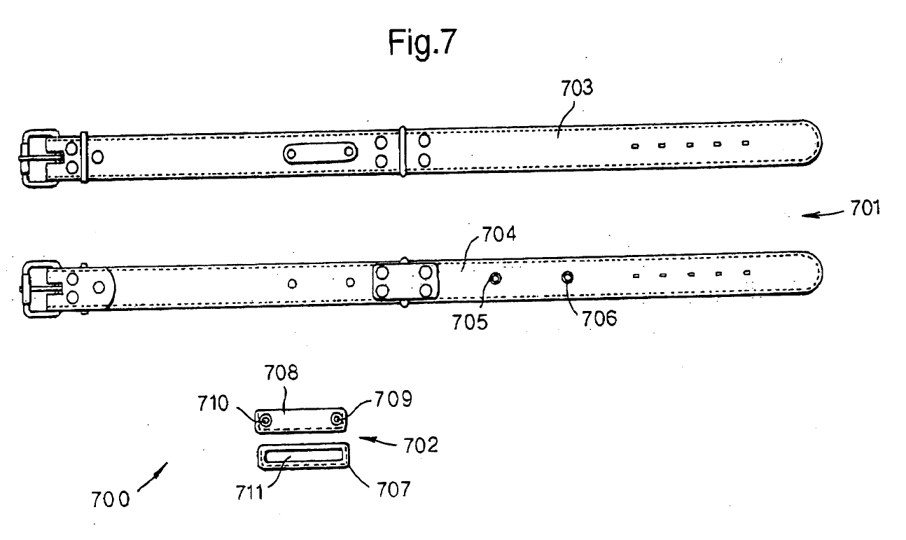

In a third specific embodiment of the present invention and referring to FIG. 4, a transdermal administration device is provided which comprises a positioning means 402, and a delivery means which comprises an impermeable substrate 403 and an adhesive matrix 404 containing substances to be administered transdermally. The positioning means 402 is a strap, and this strap comprises an outer surface 405 and an inner surface 406. The inner surface 406 of the strap 402 further comprises two female studs positioned approximately 4 centimeters apart and located in the middle of the length of the strap. The strap 402 is constructed of materials much as described for the first specific embodiment of the present invention. The impermeable substrate components 403 of the delivery means is characterized in having an outer surface 409 and an inner surface 410. The outer surface 409 of the impermeable substrate further comprises two male studs 411 and 412 spaced approximately 4 centimeters apart and configured so as to demountably attach the impermeable substrate 403 to the strap 402 via the female studs 407 and 408. This impermeable substrate may be manufactured from any material which is impervious to the substances which are to be transdermally administered and are incorporated into the adhesive matrix 404. Suitable materials include, PVC, Polypropylene, leather or any other non porous substance. The adhesive matrix 404 comprises an outer surface 413 and an inner surface 414. In this specific embodiment of the present invention this adhesive matrix comprises acrylate emulsion adhesive, but may be selected from any of the group:

acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin.

This adhesive matrix 404 is configured such the outer surface 413 of the adhesive matrix adheres to the inner surface 410 of the impermeable substrate and is fixed and held in this position. The adhesive matrix 404 further comprises a gauze material on the lower surface 414 of the adhesive matrix, this gauze material is a cotton lint in this specific embodiment but may be any form of water absorbent material to which the adhesive may adhere. Alternatively according to a further aspect of the this third specific embodiment of the present invention the adhesive matrix may comprise the adhesive selected from the group as previously described and the substance to be administered transdermally. The substances being incorporated into the adhesive during manufacture of the adhesive matrix. Alternatively, according to a further aspect of this third specific embodiment of the present invention the adhesive matrix may comprise the adhesive selected from the group as previously described and the substance to be transdermally administered in the form of a gel as previously described for the first specific embodiment of the present invention.

In this third specific embodiment of the present invention the transdermal administration device 401 may be assembled and used in the following way: assuming the strap 402 is placed upon a flat working surface with the outer surface 405 of the strap in contact with this working surface, the impermeable substrate 403 may be affixed to the strap 402 via the combination of the male and female studs 407, 408/411, 412. The outer surface 413 of the adhesive matrix 404 may then be adhered to the inner surface 410 of the impermeable substrate 403. Presenting the impregnated gauze 414, the assembled transdermal delivery device 401 may then be fitted about the animal in a fashion similar to that described previously for the first specific embodiment of the present invention. Once the adhesive matrix has been in contact with the animal for the appropriate number of days, the impermeable substrate 403 may be removed from the strap 402, the adhesive matrix 404 then being simply removed from the impermeable substrate and replaced with a fresh adhesive matrix to continue the transdermal administration of substances to the animal.

Referring to FIG. 5 herein, there are provided two schematic representations of two adhesive matrices, 501 is a schematic representation of an adhesive matrix which comprises a layer of gauze and 502 is a schematic representation of an adhesive matrix which does not comprise a layer of gauze. The first adhesive matrix 501 comprises and outer 503 and an inner 504 surface, a layer of removable material 505 forms the outer surface 503. Underneath this layer of removable material 505 is a layer of adhesive 506. This layer of adhesive may comprise any of the following:

acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin.

Underneath the layer of adhesive 506 is a layer of gauze material 507, this gauze material may be any form of water absorbent material to which the adhesive may adhere. Finally, there is a second layer of removable material 508 which comprises the lower surface 504 of this first adhesive matrix 501. This second layer of removable material 508 prevents the exposure of the gauze material 507 which has been impregnated with the substances which are to be administered transdermally. Both layers of removable material 605 and 508 are removed immediately prior to the adhesion of this adhesive matrix 501 to the impermeable substrate 403. A second form of adhesive matrix is illustrated schematically herein 502, this second adhesive matrix comprises an outer surface 509 and an inner surface 510. The outer surface 509 comprises a layer of removable material 511, beneath this layer of removable material 511 is a layer of adhesive 512. This layer of adhesive 512 comprises an adhesive selected from a group:

acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin.

This layer of adhesive 512 further comprises the substance to be administered transdermally. Beneath this adhesive layer 512 is a second layer of removable material 513 which comprises the lower surface 510 of this adhesive matrix 502. This adhesive matrix may be used in the following way. The first piece of moveable material 511 is peeled away from the adhesive layer 512, this exposed adhesive layer is then attached to the inner surface of an impermeable substrate such as that illustrated in FIG. 4. The second piece of removable material 513 may then be removed exposing the active ingredient contained within the adhesive to the environment. The impermeable substrate may then be fitted into the positioning means and the position means may be fitted about the animal bringing the substances to be transdermally administered in close contact with the animals skin.

In further specific aspects of this particular embodiment of the present invention the adhesive matrix may be impregnated with colostrum, a derivative of colostrum or a multi-vitamin and mineral solution much as previously described for the first and second specific embodiments of this invention.

In a fourth specific embodiment of the present invention, a transdermal administration device 600 is provided which comprises a collar 601 and a delivery means 602. The collar 601 further comprises an outer surface 603 and an inner surface 604. The inner surface 604 of the collar 601 comprises two female studs 605 and 606 spaced approximately 4 centimeters apart along the length of the collar 601. The collar further comprises a series of holes and a buckle such that the collar can be secured around the neck of an animal. The delivery means 602 is further characterized in that it has an outer surface 607 and an inner surface 610, the outer surface 607 comprising two male studs 608 and 609 spaced approximately 4 centimeters apart and configured such that male studs 608 and 609 may be de-mountably fixed upon collar 601 at female studs 605 and 606. The delivery means 602 being further characterized in that the lower surface 610 comprises a permeable region 611 through which substances to be transdermally administered may pass.

In use the delivery means 602 is fixed upon the collar 601 such that permeable window 611 is displayed. The collar 601 may then be fitted around the neck of an animal such that the delivery means 602 is in contact with the skin of the animal. This collar may be constructed of any impermeable material although in this specific embodiment the collar is constructed of leather coated with a layer of UPVC.

In a further aspect of this fourth specific embodiment of the present invention the delivery means 602 may be impregnated with colostrum, a derivative of colostrum or a multi-vitamin and mineral solution much as previously described for the first specific embodiment of the present invention.

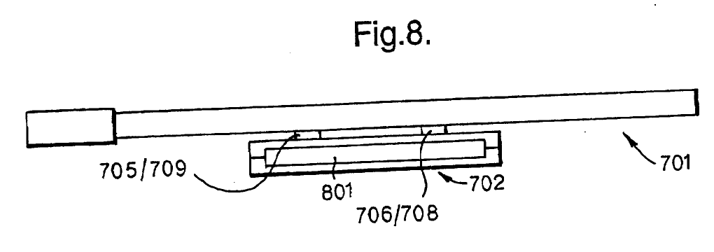

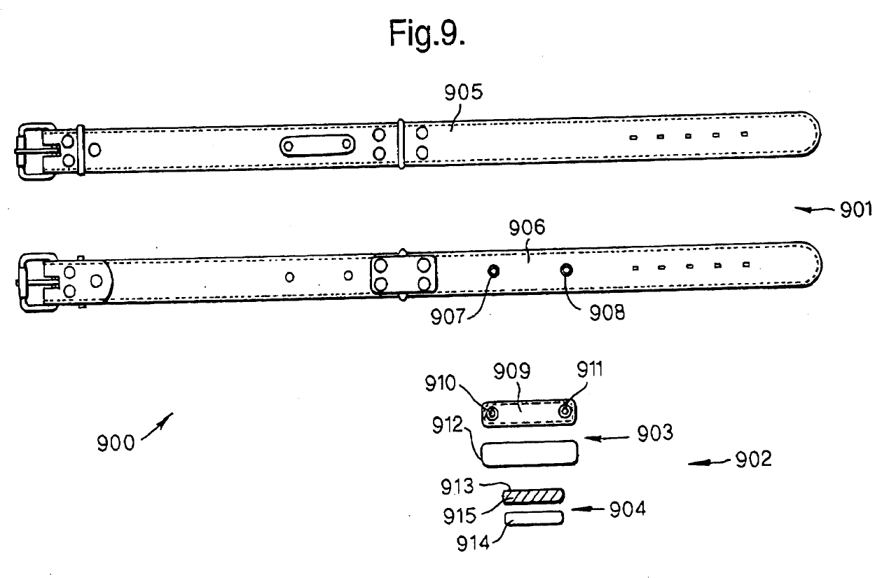

In a fifth specific embodiment of the present invention, a transdermal administration device 700 is provided which comprises a collar 701 and a delivery means 702. The collar 701 further comprises an outer surface 703 and an inner surface 704, the inner surface 704 further comprising two female studs 705 and 706 spaced approximately 4 centimeters from each other. The collar 701 further comprising a series of holes and a buckle to secure the collar 701 in place about the neck of an animal. The delivery means 702 comprises an inner surface 707 and an outer surface 708. The outer surface 708 of the delivery means 702 being further characterized in that it has two male studs 709 and 710 spaced approximately 4 centimeters from each other and being configured so as to be dismountably fixed upon the collar 701 when used with female studs 705 and 706. The inner surface 707 of the delivery means 702 further comprises a permeable window 711 through which substances are transdermally administered. Referring to FIG. 8 which shows a top down view of this fifth specific embodiment of the present invention, the delivery means 702 is further characterized in that the delivery means 702 is configured to form a reservoir 801 between the inner surface 707 and outer surface 708 of the delivery means. This reservoir 801 is configured to accept an impregnated cassette, comprising substances which are to be transdermally administered.

This fifth embodiment of the present invention may be assembled and used in the following way: the collar 701 may be placed upon a work surface with the outer surface 703 being in contact with this work surface. The delivery means 702 may then be mounted upon the collar 701 via the female and male studs 705, 706/709, 710. An impregnated cassette may then be inserted into the reservoir 801 in which it will be securely positioned and the assembled transdermal administration means may be fitted about the neck of an animal much as previously described for the fourth specific embodiment of the present invention. At the end of the service life for the impregnated cassette contained within the reservoir 801, the impregnated cassette may be removed or alternatively the complete delivery means may be removed from attachment to the collar and a new impregnated cassette may be introduced into the reservoir. Delivery means 702 may then be remounted upon the collar 701 such that impermeable window 711 is brought back into contact with the animals skin and the transdermal administration of substances may continue.

In a further aspect of the present invention the impregnated cassette may comprise colostrum, a derivative of colostrum or a multi-vitamin and mineral solution much as previously described for the first specific embodiment of the present invention.

In a sixth specific embodiment of the present invention a transdermal administration device 900 is provided which comprises a collar 901 and a delivery means 902. The delivery means 902 further comprises an impermeable substrate 903 and an adhesive matrix 904 comprising such substances as are to be administered transdermally. The collar 901 comprises an outer surface 905 and an inner surface 906, the inner surface 906 further comprising two female studs 907 and 908 spaced approximately 4 centimeters apart and positioned in the approximate middle of the collar 901 length. The collar 901 further comprises a series of holes and a buckle so as to position and hold in place the collar when in use. The impermeable substrate 903 comprises an outer surface 909 which comprises two male studs 910 and 911 spaced approximately 4 centimeters apart and configured so as to be demountably fixed upon the collar 901 via the female studs 907 and 908. The impermeable substrate further comprises an inner surface 912. The adhesive matrix comprises an inner surface 913 and an outer surface 914. This adhesive matrix may comprise an adhesive selected from the group:

acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and in this specific embodiment is an acrylic ester copolymer.

In this specific embodiment the lower surface 913 of the adhesive matrix further comprises a layer of cotton lint 915 but this may be replaced by another form of absorbent material. The substances to be administered transdermally are impregnated into this gauze material. Alternatively the adhesive matrix may comprise the substances to be administered transdermally simply in combination with the adhesive.

In this sixth preferred embodiment of the present invention the transdermal administration device may be assembled and used in the following way:

if the collar 901 is placed upon a work surface with the outer surface 905 of the collar 901 in contact with this work surface the impermeable substrate 903 may then be mounted upon the collar via the male and female studs 910, 911/907, 908 respectively. The outer surface 914 of the adhesive matrix 904 may then be applied to the inner surface 912 of the impermeable substrate affixing the adhesive matrix 904 to the impermeable substrate 903 the assembled transdermal administration device 900 may then be fitted about the neck of the animal much as previously described before for previous embodiments of the present invention. At the end of the adhesive matrix's service life the impermeable substrate 903 may simply be removed from the collar 901 and a new adhesive matrix may be applied to the inner surface 912 of the impermeable substrate 903. The impermeable substrate 903 can then be reaffixed to the collar and the transdermal administration of the required substance can continue.

According to a further aspect of this sixth specific embodiment of the present invention the adhesive matrix may be impregnated with colostrum, a colostrum derivative or a multi-vitamin and mineral solution much as previously described for other specific embodiments of the present invention.

In other embodiments of the present invention the transdermal administration device may comprise a harness in place of a strap or collar and a delivery means. All the types of delivery means as discussed in the previous six preferred embodiments of the present invention may equally be used with a harness and for the sake of clarity further explanations of the working of such embodiments are not included in the specification although they are within the invention as claimed by the applicants in this patent application.

1. A device for the transdermal administration of a substance to an animal comprising:
positioning means and delivery means:
said positioning means comprising an inner and an outer surface and capable of being configured to position and hold in place said delivery means;
said delivery means comprising an inner and an outer surface and capable of being configured to facilitate the transdermal administration of substances;
said outer surface of said delivery means and said inner surface of said positioning means are attachable to each other.

2. A device as claimed in claim 1, wherein said positioning means comprises a strap.

3. A device as claimed in claim 1, wherein said positioning means comprises a strap and said delivery means comprises a replaceable impregnated cassette.

4. A device as claimed in claim 1, wherein said positioning means comprises a strap and said delivery means comprises a replaceable cassette impregnated with colostrum.

5. A device as claimed in claim 1, wherein said positioning means comprises a strap and said delivery means comprises a replaceable cassette impregnated with a colostrum derivative.

6. A device as claimed in claim 1, wherein said positioning means comprises a strap and said delivery means comprises a replaceable cassette impregnated with a multi-vitamin and mineral solution.

7. A device as claimed in claim 1, wherein said positioning means comprises a strap and wherein said delivery means is configured to form a reservoir when held in place by said positioning means;
said reservoir being configured to accept an impregnated cassette.

8. A device as claimed in claim 1, wherein said positioning means comprises a strap and wherein said delivery means is configured to form a reservoir when held in place by said positioning means;
said reservoir being configured to accept an impregnated cassette;
wherein said cassette is impregnated with colostrum.

9. A device as claimed in claim 1, wherein said positioning means comprises a strap and wherein said delivery means is configured to form a reservoir when held in place by said positioning means;
said reservoir being configured to accept an impregnated cassette;
wherein said cassette is impregnated with a colostrum derivative.

10. A device as claimed in claim 1, wherein said positioning means comprises a strap and wherein said delivery means is configured to form a reservoir when held in place by said positioning means;
said reservoir being configured to accept an impregnated cassette;
wherein said cassette is impregnated with a multi-vitamin and mineral solution.

11. A device as claimed in claim 1, wherein said positioning means comprises a strap and said delivery means comprise an impermeable substrate and an adhesive matrix comprising said substances;
wherein said adhesive matrix is configured to adhere to the lowest surface of said impermeable substrate and the outer surface of said impermeable substrate is configured to adhere to the inner surface of said positioning means

12. A device as claimed in claim 1, wherein said positioning means comprises a strap and said delivery means comprise an impermeable substrate and an adhesive matrix comprising said substances;
wherein said adhesive matrix is configured to adhere to the lowest surface of said impermeable substrate and the outer surface of said impermeable substrate is configured to adhere to the inner surface of said positioning means;
wherein said adhesive matrix is impregnated with colostrum.

13. A device as claimed in claim 1, wherein said positioning means comprises a strap and said delivery means comprise an impermeable substrate and an adhesive matrix comprising said substances;
wherein said adhesive matrix is configured to adhere to the lowest surface of said impermeable substrate and the outer surface of said impermeable substrate is configured to adhere to the inner surface of said positioning means;
wherein said adhesive matrix is impregnated with a colostrum derivative.

14. A device as claimed in claim 1, wherein said positioning means comprises a strap and said delivery means comprise an impermeable substrate and an adhesive matrix comprising said substances;
wherein said adhesive matrix is configured to adhere to the lowest surface of said impermeable substrate and the outer surface of said impermeable substrate is configured to adhere to the inner surface of said positioning means;
wherein said adhesive matrix is impregnated with multi-vitamin and mineral solution,

15. A device as claimed in claim 11 or claim 12 or claim 13, wherein said adhesive is selected from the group comprising:
accrylate emulsion adhesive, an acrylic ester copolymer, a vinyl acetate resin, a copolymer of vinyl acetate and dioctyl maleate, silicon adhesive, natural or synthetic rubber, a petroleum derivative, a resin.

16. A device as claimed in claim 1, wherein said positioning means comprises a collar.

17. A device as claimed in claim 1 wherein said positioning means comprises a collar and said delivery means comprises a replaceable impregnated cassette.

18. A device as claimed in claim 1, wherein said positioning means comprises a collar and said delivery means comprises a replaceable cassette impregnated with colostrum.

19. A device as claimed in claim 1, wherein said positioning means comprises a collar and said delivery means comprises a replaceable cassette impregnated with a colostrum derivative.

20. A device as claimed in claim 1, wherein said positioning means comprises a collar and said delivery means comprises a replaceable cassette impregnated with a multi-vitamin and mineral solution.

21. A device as claimed in claim 1, wherein said positioning means comprises a collar and wherein said delivery means is configured to form a reservoir when held in place by said positioning means;
said reservoir being configured to accept an impregnated cassette.

22. A device as claimed in claim 1, wherein said positioning means comprises a collar and wherein said delivery means is configured to form a reservoir when held in place by said positioning means;
said reservoir being configured to accept an impregnated cassette;
wherein said cassette is impregnated with colostrum.

23. A device as claimed in claim 1, wherein said positioning means comprises a collar and wherein said delivery means is configured to form a reservoir when held in place by said positioning means;
said reservoir being configured to accept an impregnated cassette;
wherein said cassette is impregnated with a colostrum derivative.

24. A device as claimed in claim 1, wherein said positioning means comprises a collar and wherein said delivery means is configured to form a reservoir when held in place by said positioning means;
said reservoir being configured to accept an impregnated cassette;
wherein said cassette is impregnated with a multi-vitamin and mineral solution.

25. A device as claimed in claim 1, wherein said positioning means comprises a collar and said delivery means comprise an impermeable substrate and an adhesive matrix comprising said substances;
wherein said adhesive matrix is configured to adhere to the lowest surface of said impermeable substrate and the outer surface of said impermeable substrate is configured to adhere to the inner surface of said positioning means.

26. A device as claimed in claim 1, wherein said positioning means comprises a collar and said delivery means comprise an impermeable substrate and an adhesive matrix comprising said substances;
wherein said adhesive matrix is configured to adhere to the lowest surface of said impermeable substrate and the outer surface of said impermeable substrate is configured to adhere to the inner surface of said positioning means;
wherein said adhesive matrix is impregnated with colostrum.

27. A device as claimed in claim 1, wherein said positioning means comprises a collar and said delivery means comprise an impermeable substrate and an adhesive matrix comprising said substances;
wherein said adhesive matrix is configured to adhere to the lowest surface of said impermeable substrate and the outer surface of said impermeable substrate is configured to adhere to the inner surface of said positioning means;
wherein said adhesive matrix is impregnated with a colostrum derivative.

28. A device as claimed in claim 1, wherein said positioning means comprises a collar and said delivery means comprise an impermeable substrate and an adhesive matrix comprising said substances;
wherein said adhesive matrix is configured to adhere to the lowest surface of said impermeable substrate and the outer surface of said impermeable substrate is configured to adhere to the inner surface of said positioning means;
wherein said adhesive matrix is impregnated with a multivitamin and mineral solution.

29. A device as claimed in claim 1, wherein said positioning means comprises a harness.

30. A device as claimed in claim 1, wherein said positioning means comprises a harness and said delivery means comprises a replaceable impregnated cassette.

31. A device as claimed in claim 1, wherein said positioning means comprises a harness and said delivery means comprises a replaceable cassette impregnated with colostrum.

32. A device as claimed in claim 1, wherein said positioning means comprises a harness and said delivery means comprises a replaceable cassette impregnated with a colostrum derivative.

33. A device as claimed in claim 1, wherein said positioning means comprises a harness and said delivery means comprises a replaceable cassette impregnated with a multi-vitamin and mineral solution.

34. A device as claimed in claim 1, wherein said positioning means comprises a harness and wherein said delivery means is configured to form a reservoir when held in place by said positioning means;
said reservoir being configured to accept an impregnated cassette.

35. A device as claimed in claim 1, wherein said positioning means comprises a harness and wherein said delivery means is configured to form a reservoir when held in place by said positioning means;
said reservoir being configured to accept an impregnated cassette;
wherein said cassette is impregnated with colostrum.

36. A device as claimed in claim 1, wherein said positioning means comprises a harness and wherein said delivery means is configured to form a reservoir when held in place by said positioning means;
said reservoir being configured to accept an impregnated cassette;
wherein said cassette is impregnated with a colostrum derivative.

37. A device as claimed in claim 1, wherein said positioning means comprises a harness and wherein said delivery means is configured to form a reservoir when held in place by said positioning means;
said reservoir being configured to accept an impregnated cassette;
wherein said cassette is impregnated with a multi-vitamin and mineral solution.

38. A device as claimed in claim 1, wherein said positioning means comprises a harness and said delivery means comprise an impermeable substrate and an adhesive matrix comprising said substances;
wherein said adhesive matrix is configured to adhere to the lowest surface of said impermeable substrate and the outer surface of said impermeable substrate is configured to adhere to the inner surface of said positioning means.

39. A device as claimed in claim 1, wherein said positioning means comprises a harness and said delivery means comprise an impermeable substrate and an adhesive matrix comprising said substances;
wherein said adhesive matrix is configured to adhere to the lowest surface of said impermeable substrate and the outer surface of said impermeable substrate is configured to adhere to the inner surface of said positioning means;
wherein said adhesive matrix is impregnated with colostrum.

40. A device as claimed in claim 1, wherein said positioning means comprises a harness and said delivery means comprise an impermeable substrate and an adhesive matrix comprising said substances;
wherein said adhesive matrix is configured to adhere to the lowest surface of said impermeable substrate and the outer surface of said impermeable substrate is configured to adhere to the inner surface of said positioning means;
wherein said adhesive matrix is impregnated with a colostrum derivative.

41. A device as claimed in claim 1, wherein said positioning means comprises a harness and said delivery means comprise an impermeable substrate and an adhesive matrix comprising said substances;
wherein said adhesive matrix is configured to adhere to the lowest surface of said impermeable substrate and the outer surface of said impermeable substrate is configured to adhere to the inner surface of said positioning means;
wherein said adhesive matrix is impregnated with a multivitamin and mineral solution.

42. A device as claimed in claim 1, wherein said delivery means comprises a replaceable impregnated cassette.

43. A device as claimed in claim 1, wherein said delivery means comprises configured to form a reservoir when held in place by said positioning means;
said reservoir being configured to accept an impregnated cassette.

44. A device as claimed in claim 1, wherein said delivery means comprises an impermeable substrate and an adhesive matrix comprising said substances;
wherein said adhesive matrix is configured to adhere to the inner surface of said impermeable substrate and the outer surface of said impermeable substrate is configured to adhere to said positioning means.

45. A device as claimed in claim 1, wherein said substances comprise colostrum.

46. A device as claimed in claim 1, wherein said substances comprise a colostrum derivative.

47. A device as claimed in claim 1, wherein said substances comprise a multi-vitamin and mineral solution.

48. A pet restraint device comprising means to transdermally administer compounds;
said means comprising a releasably mounted plate.

49. A pet restraint device as claimed in claim 48, wherein said pet restraint device is a collar.

50. A pet restraint device as claimed in claim 48, wherein said pet restraint device is a harness.

51. A pet restraint device as claimed in claim 48, wherein said compounds comprise colostrum.

52. A pet restraint device as claimed in claim 48, wherein said releasably mounted plate comprises a colostrum derivative.

53. A pet restraint device as claimed in claim 48, wherein said releasably mounted plate comprises a multi-vitamin and mineral solution.

54. A pet restraint device as claimed in claim 48, wherein said releasably mounted plate is configured to form a compartment when mounted upon said pet restraint device;
said compartment being configured to hold a permeable cassette containing said compounds.

55. A pet restraint device as claimed in claim 48, wherein said device further comprises an adhesive backed pad;
said adhesive backed pad being configured to adhere to said releasably mounted plate;
wherein said adhesive backed pad comprises said compounds
